(19) United States
(12) Patent Application Publication
(10) Pub. No.: US 2008/0113037 A1
(43) Pub. Date: May 15, 2008

(54) Topical Compositions Comprising Polyhydroxy Acids and/or Lactones for Improved Cutaneous Effects of Oxidative Therapeutic Drugs

(76) Inventors: Barbara A. Green, Edison, NJ (US); David J. Milora, Blue Bell, PA (US)

Correspondence Address: GOODWIN PROCTER LLP, 901 NEW YORK AVENUE, N.W., WASHINGTON, DC, 20001, US

(21) Appl. No.: 11/682,039

(22) Filed: Mar. 5, 2007

Related U.S. Application Data
(60) Provisional application No. 60/865,244, filed on Nov. 10, 2006.

(51) Int. Cl. A61K 33/40; A61K 31/34; A61K 31/385; A61K 33/04; A61K 47/12; A61P 17/00; A61K 35/04; A61K 33/22; A61K 33/32

(52) U.S. Cl. 424/616; 424/640; 424/660; 424/667; 424/703; 424/725.1; 514/18; 514/356; 514/440; 514/460; 514/714; 514/785

(57) ABSTRACT

Embodiments relate generally to topical compositions useful for treating or preventing a variety of cosmetic conditions and dermatological disorders, where the composition comprises an oxidative pharmaceutical drug and an antioxidant polyhydroxy acid or polyhydroxy lactone. The polyhydroxy acid or polyhydroxy lactone improves the cutaneous effects of the drug. The embodiments further relate to the treatment of a variety of cosmetic conditions and dermatological disorders comprising administering to a subject an effective amount of the composition and to methods of making the compositions.

### CROSS-REFERENCE TO RELATED APPLICATION

This application claims benefit of U.S. Provisional Application No. 60/865,244, filed on Nov. 10, 2006, which is hereby incorporated by reference in its entirety.

### FIELD OF THE INVENTION

Embodiments relate generally to topical compositions useful for treating or preventing a variety of cosmetic conditions and dermatological disorders, where the composition comprises an oxidative pharmaceutical drug and a polyhydroxy acid or polyhydroxy lactone. The polyhydroxy acid or polyhydroxy lactone improves the cutaneous effects of the drug. The embodiments further relate to the treatment of variety of cosmetic conditions and dermatological disorders comprising administering to a subject an effective amount of the composition and to methods of making the compositions.

### DESCRIPTION OF RELATED ART

Oxidative pharmaceutical drugs can be chemical compounds that contain at least one oxidative functional group or that function as oxidative substances when topically applied to the skin, such as peroxide, peracid, superoxide and the like. Examples of such drugs are benzoyl peroxide, hydrogen peroxide and perbenzoic acid.

When an oxidative pharmaceutical drug is administered to cutaneous tissues including the skin for topical treatment of dermatological disorders, the adverse reactions to the skin include (a) dryness, (b) irritation, (c) erythema, (d) damage and (e) other miscellaneous side effects. Such adverse reactions and damages to the skin are not required for or are unrelated to the therapeutic effects of the drug.

U.S. Pat. No. 6,036,963 discloses the use of gluconolactone or glucarolactone in cosmetic skin care compositions as an anti-irritant. The gluconolactone or glucarolactone are said to alleviate or control any skin irritation that may be caused by glycolic acid.

### SUMMARY OF INVENTION

It is desirable to minimize or eliminate the adverse reactions and damages to the skin caused by oxidative pharmaceutical drugs. These adverse reactions are not required for or are unrelated to the therapeutic effects of the drug. The present inventors have discovered that when a polyhydroxy acid or polyhydroxy lactone is incorporated into a composition comprising the oxidative pharmaceutical drug, the adverse reactions to the skin by the drugs are eliminated or minimized.

One embodiment of this invention is a composition for topical administration comprising an oxidative pharmaceutical drug and a polyhydroxy acid or polyhydroxy lactone. Another embodiment of this invention is a method of topical cutaneous treatment of a variety of cosmetic conditions and dermatological disorders comprising administering to a subject an effective amount of a composition comprising an oxidative pharmaceutical drug and a polyhydroxy acid or polyhydroxy lactone. Another embodiment of this invention is a method of making a composition for topical administration that can be useful for treating a variety of cosmetic conditions and dermatological disorders, where the composition comprises an oxidative pharmaceutical drug and a polyhydroxy acid or polyhydroxy lactone.

### DETAILED DESCRIPTION OF THE PREFERRED EMBODIMENTS

The terminology used herein is for the purpose of describing particular embodiments only, and is not intended to limit the scope of the present invention. As used throughout this disclosure, the singular forms “a,” “an,” and “the” include plural references unless the context clearly dictates otherwise.

Oxidative pharmaceutical drugs can be chemical compounds that contain at least one oxidative functional group or that function as oxidative substances when topically applied to the cutaneous tissue, such as peroxide, peracid, superoxide and the like. Examples of such drugs are benzoyl peroxide, carbamide peroxide, coal tar, glutathione, hydrogen peroxide, iodine, juniper tar, lipoic acid, NAD⁺, NADP⁺, nitrogen oxide, oxygen, pine tar, potassium permanganate, povidone-iodine, perbenzoic acid, sulfur, sulfur dioxide, sodium borate, sodium perborate, shale tar, ubiquinone and wood tar.

When an oxidative pharmaceutical drug is administered to cutaneous tissues including the skin for topical treatment, adverse reactions occur. The adverse reactions to the skin include (a) dryness, (b) irritation, (c) erythema, (d) damage and (e) other miscellaneous side effects. These adverse reactions are not required for or are unrelated to the therapeutic effects of the drug. The present inventors have discovered that when a polyhydroxy acid or polyhydroxy lactone is incorporated into a composition comprising the oxidative pharmaceutical drug, the adverse reactions to the skin by the drug are eliminated or minimized, and therapeutic efficacy is maintained or enhanced.

As an illustration, when a composition containing oxidative benzoyl peroxide is administered for topical treatment of acne, the skin becomes dry, erythematous and irritated. Many subjects discontinue the use of such compositions because of the adverse skin reactions. When a composition comprising gluconolactone and oxidative benzoyl peroxide is administered topically, however, there is less, or even no irritation, and the acne lesions are improved.

Polyhydroxy acids and polyhydroxy lactones preferably are (a) modulators for skin keratinization, (b) antioxidant compounds, (c) preventive or counter-irritants, (d) moisturizers, (e) skin barrier strengtheners, (f) restorers when topically administered to cutaneous tissues; and (g) anti-aging compounds by increasing dermal biosynthesis. Polyhydroxy acids and polyhydroxy lactones typically are organic carboxylic compounds having at least two hydroxyl groups in the molecules and with preferred molecular weight of between about 100 and about 300. These polyhydroxy acids and polyhydroxy lactones include gluconic acid, gluconolactone, ribonic acid, ribonolactone, galactonic acid, galactonolactone, glucoheptonic acid, glucoheptonolactone, glucuronic acid, glucuronolactone, galacturonic acid, galacturonolactone, glucaric acid, glucarolactone, galactaric acid and galactarolactone.

The polyhydroxy acids and polyhydroxy lactones of the present embodiments can be divided into the following three groups.

(A) Aldonic Acids and Aldonolactones

When a common carbohydrate, also called aldose, is oxidized at the carbon one position from an aldehyde to a carboxyl group, the product is called aldonic acid. For example, when glucose is oxidized at the carbon one position, the product is gluconic acid. The aldonic acid usually has multiple hydroxyl groups. The generic structure can be shown as follows:

R(CHOH)ₙCHOH COOH

where R is usually H or an alkyl group and n is an integer from 1-9. The aldonic acids can exist as stereoisomers as D, L and DL or R, S and RS forms. Many aldonic acids form intramolecular lactones, aldonolactones, by removing one mole of water between the carboxyl group and one hydroxyl group.

The following are representative aldonic acids and aldonolactones: 2,3-dihydroxypropanoic acid (glyceric acid); 2,3,4-trihydroxybutanoic acids (stereoisomers; erythronic acid and erythronolactone, threonic acid and threonolactone); 2,3,4,5-tetrahydroxypentanoic acids (stereoisomers; ribonic acid and ribonolactone, arabinoic acid and arabinolactone, xylonic acid and xylonolactone, lyxonic acid and lyxonolactone); 2,3,4,5,6-pentahydroxyhexanoic acids (stercoisomers; allonic acid and allonolactone, altronic acid and altronolactone, gluconic acid and gluconolactone, mannoic acid and mannolactone, gulonic acid and gulonolactone, idonic acid and idonolactone, galactonic acid and galactonolactone, talonic acid and talonolactone); 2,3,4,5,6,7-hexahydroxyheptanoic acids (stereoisomers; alloheptonic acid and alloheptonolactone, altroheptonic acid and altroheptonolactone, glucoheptonic acid and glucoheptonolactone, mannoheptonic acid and mannoheptonolactone, guloheptonic acid and guloheptonolactone, idoheptonic acid and idoheptonolactone, galactoheptonic acid and galactoheptonolactone, taloheptonic acid and taloheptonolactone).

(B) Aldaric Acids and Aldarolactones.

The aldaric acid typically has multiple hydroxyl groups attached to the carbon chain surrounded by two carboxyl groups. Many aldaric acids form intramolecular lactones, aldarolactones, by removing one mole of water between one of the two carboxyl groups and one hydroxyl group, such as glucarolactone from glucaric acid. The generic structure can be shown as follows:

HOOC(CHOH)ₙCHOH COOH

where n is an integer from 1-9. The aldaric acids can exist as stereoisomers as D, L and DL or R, S and RS forms.

The following are representative aldaric acids and aldarolactones: 2,3-dihydroxybutane-1,4-dioic acids (stereoisomers; erythraric acid and threaric acid); 2,3,4-trihydroxypentane-1,5-dioic acids (stereoisomers; ribaric acid and ribarolactone, arabaric acid and arabarolactone, xylaric acid and xylarolactone, lyxaric acid and lyxarolactone); 2,3,4,5-tetrahydroxyhexane-1,6-dioic acids (stereoisomers; allaric acid and allarolactone, altraric acid and altrarolactone, glucaric acid and glucarolactone, mannaric acid and mannarolactone, gularic acid and gularolactone, idaric acid and idarolactone, galactaric acid and galactarolactone, talaric acid and talarolactone); 2,3,4,5,6-pentahydroxyheptane-1,7-dioic acids (stereoisomers; alloheptaric acid and alloheptarolactone, altroheptaric acid and altroheptarolactone, glucoheptaric acid and glucoheptarolactone, mannoheptaric acid and mannoheptarolactone, guloheptaric acid and guloheptarolactone, idoheptaric acid and idoheptarolactone, galactoheptaric acid and galactoheptarolactone, taloheptaric acid and taloheptarolactone).

(C) Alduronic Acids and Alduronolactones.

Alduronic acid is typically obtained from a carbohydrate, aldose, by oxidation of the terminal carbon to carboxyl group, and the carbon one position remains as aldehyde group, such as glucuronic acid from glucose. Similar to aldonic acid and aldaric acid, alduronic acid also has multiple hydroxyl groups attached to the carbon chain between two functional groups, one aldehyde and one carboxyl groups in this case. Many alduronic acids exist as intramolecular lactones, alduronolactones, such as glucuronolactone from glucuronic acid. The generic structure can be shown as follows:

HOOC(CHOH)ₙCHOH CHO

where n is an integer from 1-9. The alduronic acids can exist as stereoisomers as D, L and DL or R, S and RS forms.

The following are representative alduronic acids and alduronolactones: erythruronic acid and threuronic acid, riburonic acid and riburonolactone, araburonic acid and araburonolactone, xyluronic acid and xyluronolactone, lyxuronic acid and lyxuronolactone, alluronic acid and alluronolactone, altruronic acid and altruronolactone, glucuronic acid and glucuronolactone, mannuronic acid and mannuronolactone, guluronic acid and guluronolactone, iduronic acid and iduronolactone, galacturonic acid and galacturonolactone, taluronic acid and taluronolactone, allohepturonic acid and allohepturonolactone, altrohepturonic acid and altrohepturonolactone, glucohepturonic acid and glucohepturonolactone, mannohepturonic acid and mannohepturonolactone, gulohepturonic acid and gulohepturonolactone, idohepturonic acid and idohepturonolactone, galactohepturonic acid and galactohepturonolactone, talohepturonic acid and talohepturonolactone.

The compositions of the present embodiments also may contain other pharmaceutical or topical agents for synergetic or synergistic effects. The pharmaceutical and other topical agents which can be incorporated into the compositions include those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; anfiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; and depilating agents.

Examples of the above agents include abacavir, acebutolol, acetaminophen, acetaminosalol, acetazolamide, acetohydroxamic acid, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithiromycin, bacitracin, beclomethasone dipropionate, bemegride, benazepril, bendroflumethiazide, benzocaine, benzonatate, benzophenone, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamnide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotarnine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosiine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolarnine, phenytoin, physostigmine, pilocarpine, pimozide, pindolol, pioglitazone, pipamazine, piperonyl butoxide, pirenzepine, podofilox, podophyllin, pratipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triarncinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, xanthine, zafirlukast, zaleplon, zinc pyrithione, ziprasidone, zolmitriptan and zolpidem.

In accordance to one embodiment of this invention, compositions comprising an oxidative pharmaceutical drug and a polyhydroxy acid or polyhydroxy lactone, can be useful for treating a variety of cosmetic conditions. Such cosmetic conditions or dermatological disorders include disturbed keratinization, defective syntheses of dermal components, and changes associated with aging of skin, nail and hair; and those indications which include dryness or loose of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbac; eczema; psoriasis; itchy scalp and skin; pruritus; warts; herpes; age spots; lentigines; melasmas; blemished skin; hyperkeratoses; hyperpigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair, skin thickening due to elastosis of photoaging, loss or reduction of skin, nail and hair resiliency, elasticity and recoilability; lack of skin, nail and hair lubricants and luster; dull and older-looking skin, nail and hair; and fragility and splitting of nail and hair.

The concentration of an oxidative pharmaceutical drug in a composition where the composition comprises an oxidative pharmaceutical drug and a polyhydroxy acid or polyhydroxy lactone, preferably is a concentration sufficient to provide the desired cosmetic or dermatological effect, which may vary depending on the desired cosmetic condition or dermatological disorder being treated, the size of the patient, and other factors. Preferably, the concentration of the oxidative pharmaceutical drug ranges from about 0.01% to about 99.9% by weight, based on the total weight of the composition, more preferably from about 0.1% to about 50% by weight, based on the total weight of the composition, and most preferably from about 0.5% to 25% by weight, based on the total weight of the composition. Those skilled in the art are capable of determining a suitable concentration of oxidative pharmaceutical drug, using the guidelines provided herein.

The concentration of a polyhydroxy acid or polyhydroxy lactone where the composition comprises an oxidative pharmaceutical drug and a polyhydroxy acid or polyhydroxy lactone, preferably is a concentration sufficient to provide the desired cosmetic or dermatological effect, which may vary depending on the desired cosmetic condition or dermatological disorder being treated, the extent and type of deleterious effects from the drug on the skin, and other factors. Clinical benefits achieved by including a polyhydroxy acid or polyhydroxy lactone in combination with an oxidizing drug will vary depending on the nature and concentration of the oxidizing drug, as well as the formulation vehicle. It is possible to eliminate or minimize the adverse reactions on skin by the oxidative drug and maintain or enhance therapeutic efficacy of the oxidative drug using a low, middle or high concentration of polyhydroxy acid and/or polyhydroxy lactone in an acceptable vehicle. Careful selection of vehicle components along with clinical screening can be used to determine the ideal combination of oxidative drug and polyhydroxy acid and/or polyhydroxy lactone. Those skilled in the art are capable of determining the appropriate combinations of components, and amounts, using the guidelines provided herein. Preferably, the concentration of the polyhydroxy acid or polyhydroxy lactone ranges from about 0.01% to about 99.9% by weight, based on the total weight of the composition, more preferably from about 0.1% to about 50% by weight, based on the total weight of the composition, and most preferably from about 0.5% to 25% by weight, based on the total weight of the composition. Those skilled in the art are capable of determining a suitable concentration of polyhydroxy acid or polyhydroxy lactone, using the guidelines provided herein.

In accordance with an embodiment of the invention, there is provided a method of making a composition for topical administration comprising an oxidative pharmaceutical drug and a polyhydroxy acid or polyhydroxy lactone. A solution comprising the polyhydroxy acid or polyhydroxy lactone is prepared, preferably with mixing and heating. Water soluble oxidative pharmaceutical drugs can be prepared in solution. An example of an insoluble oxidative pharmaceutical drug, specifically benzoyl peroxide, is prepared using a dispersion of the oxidative pharmaceutical drug as described herein. The particle size of the oxidative pharmaceutical drug is reduced to an appropriate distribution where the dispersion contains minimal grittiness and minimal discernable feel when applied to the skin. This is achieved by wet milling, roller milling, crushing, or other typical methods of reducing particle sizes. The solution comprising the polyhydroxy acid or polyhydroxy lactone is mixed with the oxidative pharmaceutical drug dispersion. If the solution comprising the polyhydroxy acid or polyhydroxy lactone was previously heated, it is first cooled prior to mixing with the oxidative pharmaceutical drug dispersion.

To prepare a topical combination composition for synergetic or synergistic effects, an additional pharmaceutical or topical agent is incorporated into the above compositions by dissolving or mixing the agent into the formulation.

The following are illustrative examples of formulations and other aspects of the present embodiments. Although the examples utilize only selected compounds and formulations, it should be understood that the following examples are illustrative and not limiting.

### EXAMPLE 1

### Benzoyl Peroxide Cream Containing Gluconolactone

The following is an example of the preparation of a benzoyl peroxide cream containing the polyhydroxy lactone, Gluconolactone, for improved cutaneous effects. This formulation yields, but is not limited to, a cream containing 10% benzoyl peroxide and 10% gluconolactone (which is subsequently hydrolyzed to gluconic acid during preparation). Other formulation percentages of both benzoyl peroxide and gluconolactone may be employed as desired.

Ingredients:

| | Ingredients | % W/W |
|---|---|---|
| Part A | Purified Water | 28.00 |
| | Magnesium Aluminum Silicate | 1.00 |
| | Glycerin | 2.00 |
| | Xanthan Gum | 0.75 |
| Part B | Purified Water | 20.00 |
| | Gluconolactone | 10.00 |
| | Strong Ammonia Solution | To desired pH |
| Part C | Cetyl Alcohol | 1.00 |
| | Dimethicone | 1.00 |
| | Glyceryl Stearate | 2.85 |
| | PEG-100 Stearate | 1.25 |
| | PEG-40 Stearate | 2.00 |
| Part D | Purified Water | 13.00 |
| | 75% Benzoyl Peroxide | 13.33 |
| | Sodium Dioctylsodiumsulfosuccinate | 0.06 |
| Enhancement | Defoaming Agent | QS |
| Additives | Preservative | QS |
| | Purified Water | QS to 100% |

Typical Preparation Method: Prepare Part A by adding water and Magnesium Aluminum Silicate to the main vessel with mixing. Add a premixed mixture of Xantham Gum and Glycerin to the mixture already in the main vessel. Prepare Part B in a separate vessel by adding water and Gluconolactone with continuous mixing. Continue mixing for a minimum of 2 hours to effect complete hydrolysis of the gluconolactone to gluconic acid. After the appropriate mixing time, adjust pH with Ammonium Hydroxide solution (or other suitable alkali) to the desired amount of free acid and pH. Add Part B to Part A and heat the Part AB mixture to 70°-75° C. Prepare Part C in a separate vessel by adding all the ingredients of Part C and heat to 70°-75° C. Start continuous mixing when the solid material begins to melt. In a separate vessel prepare Part D. Reduce the particle size of the Benzoyl Peroxide dispersion to an appropriate distribution where the dispersion contains minimal grittiness when applied to the skin. This is achieved by any of the typical methods such as wet milling, roller milling, or crushing to achieve minimal discernable feel on the skin. Prepare the emulsion by adding and mixing continuously the Part C mixture to the Part AB mixture when all the mixtures are at 70°-75° C., Cool the emulsion to below 40° C. and add the Part D Benzoyl Peroxide dispersion with continuous mixing. Homogenize the mixture if desired. Add the remaining optional ingredients and sufficient water to replace losses incurred during preparation.

### EXAMPLE 2

### Benzoyl Peroxide Low Viscosity Fluid Containing Gluconolactone

The following is an example of the preparation of a benzoyl peroxide low viscosity fluid, typically used for application from a pad, swab, dauber, or other similar device. This formulation contains the polyhydroxy lactone, Gluconolactone, for improved cutaneous effects. This formulation yields, but is not limited to, a low viscosity fluid containing 7.5% benzoyl peroxide and 10% gluconolactone (which is subsequently hydrolyzed to gluconic acid during preparation). Other formulation percentages of both benzoyl peroxide and Gluconolactone may be employed as desired.

Ingredients:

| | Ingredients | % W/W |
|---|---|---|
| Part A | Purified Water | 18.00 |
| | Magnesium Aluminum Silicate | 1.00 |
| | Glycerin | 5.00 |
| | Xanthan Gum | 0.15 |
| Part B | Purified Water | 20.00 |
| | Gluconolactone | 10.00 |
| | Strong Ammonia Solution | To desired pH |
| Part C | Cetyl Alcohol | 0.50 |
| | Dimethicone | 3.00 |
| | Steareth-20 | 2.31 |
| | Steareth-2 | 0.68 |
| Part D | Purified Water | 27.00 |
| | 75% Benzoyl Peroxide | 10.00 |
| | Cetyl Alcohol | 0.75 |
| Enhancement | Defoaming Agent | QS |
| Additives | Preservative | QS |
| | Purified Water | QS to 100% |

Typical Preparation Method: See example 1 for a typical preparation method.

### EXAMPLE 3

### Benzoyl Peroxide Creamy Cleanser Containing Gluconolactone

The following is an example of the preparation of a benzoyl peroxide creamy cleanser containing the polyhydroxy lactone, Gluconolactone, for improved cutaneous effects. This formulation yields, but is not limited to, a creamy cleanser containing 7.5% benzoyl peroxide and 10% gluconotactone. Other formulation percentages of both benzoyl peroxide and gluconolactone may be employed as desired.

Ingredients:

| | Ingredients | % W/W |
|---|---|---|
| Part A | Purified Water | 25.00 |
| | Gluconolactone | 10.00 |
| | Strong Ammonia Solution | To desired pH |
| | Glycerin | 1.00 |
| | Xanthan Gum | 0.25 |
| | Magnesium Aluminum Silicate | 0.45 |
| | Cetyl Alcohol | 6.00 |
| | Cocoamidopropyl Betaine | 8.00 |
| | Cetearyl Alcohol | 1.00 |
| Part B | Purified Water | 27.00 |
| | 75% Benzoyl Peroxide | 13.33 |
| | Cetyl Alcohol | 1.00 |
| | 75% Benzoyl Peroxide | 13.33 |
| | Sodium Dioctylsodiumsulfosuccinate | 0.06 |
| Enhancement | Defoaming Agent | QS |
| Additives | Preservative | QS |
| | Purified Water | QS to 100% |

Typical Preparation Method: Prepare Part A by dissolving gluconolactone in water. When dissolved, add a portion of the Cocoamidopropyl Betaine and mix for approximately 2 hours. Adjust to desired pH and add the Magnesium Aluminum Silicate. Premix the Xanthan Gum with the Glycerin and add to the previous mixture. Heat to 60°-70°C. Add the remaining Part A ingredients and heat to 70°-75° C. forming a smooth mixture with all the solid ingredients melted. Cool the emulsion to below 40° C. and add the Part B Benzoyl Peroxide dispersion with continuous mixing. Homogenize if desired. Add the remaining optional ingredients and sufficient water to replace any losses incurred during preparation.

STABILITY DATA: An analysis of the stability of the compositions in examples 1-4 using HPLC yielded the following results.

Stability Data of Examples 1-3

% Benzoyl Peroxide Analyzed by HPLC

| | Initial | 4-Weeks | 8-Weeks | 13-Weeks |
|---|---|---|---|---|
| Example 1 | | | | |
| 25° C. | 10.0% | 9.5% | 9.6% | 9.6% |
| 30° C. | 10.0% | 9.4% | 9.4% | 9.6% |
| 40° C. | 10.0% | 9.4% | 9.3% | 9.3% |
| Example 2 | | | | |
| 25° C. | 7.7% | 7.8% | 7.9% | 7.8% |
| 30° C. | 7.7% | 7.8% | 7.8% | 7.8% |
| 40° C. | 7.7% | 7.7% | 7.5% | 7.1% |
| Example 3 | | | | |
| 25° C. | 7.7% | 7.8% | — | 7.8% |
| 30° C. | 7.7% | 7.8% | — | 7.9% |
| 40° C. | 7.7% | 7.9% | — | 7.0% |

### EXAMPLE 4

### Benzoyl Peroxide Invert Gel Containing Gluconolactone

The following is an example of the preparation of a benzoyl peroxide invert gel containing the polyhydroxy lactone, Gluconolactone, for improved cutaneous effects. This formulation yields, but is not limited to, a water-in-silicone gel containing 10% benzoyl peroxide and 5% gluconolactone (which is subsequently hydrolyzed to gluconic acid during preparation). Other formulation percentages of both benzoyl peroxide and gluconolactone may be employed as desired.

Ingredients:

| | Ingredients | % W/W |
|---|---|---|
| Part A | Dimethicone Copolyol | 1.00 |
| | Cyclomethicone | 16.00 |
| | Cyclomethicone/Dimethiconol | 3.00 |
| Part B | Purified Water | 31.00 |
| | Gluconolactone | 10.00 |
| | Propylene Glycol | 8.00 |
| | Strong Ammonia Solution | To desired pH |
| Part C | Purified Water | 14.00 |
| | 75% Benzoyl Peroxide | 13.33 |
| | Sodium Dioctylsodiumsulfosuccinate | 0.06 |
| Enhancement | Preservative | QS |
| Additives | Purified Water | QS to 100% |

Typical Preparation Method: Prepare Part A by blending all the ingredients of Part A. Prepare Part B by blending all the ingredients of Part B as described in Example 1 and adjust to the desired pH with an alkaline base. Enhancement additives are added as desired. Reduce particle size of Part C to appropriate distribution as described in Example 1. Mix Part B and C together and then add the Part BC mixture to Part A with mixing to form a water-in-silicone gel.

### EXAMPLE 5

### Polyhydroxy Acid and Polyhydroxy Bionic Acid—Thritation Potential

A 14-day cumulative irritation study was conducted on 24 healthy subjects to evaluate irritation potential of a commercially available polyhydroxy acid product containing 4% lactobionic acid +8% gluconolactone cream (pH 3.8). The product was tested in comparison to a mild irritant (0.1% sodium lauryl sulfate solution) and a negative control (0.9% sodium chloride solution (saline)). Test products were applied under full occlusion for days 1 to 14, and observations were made daily including weekends. The polyhydroxy acid cream scored statistically equivalently in irritation potential to normal saline, and significantly less than the sodium lauryl sulfate irritant control. Accordingly, polyhydroxy acids and polyhydroxy bionic acids by themselves are not irritating compounds, although prior to the present disclosure, it was not known that these compounds could reduce the irritating effects and potential of oxidative drugs.

### EXAMPLE 6

### Benzoyl Peroxide+Polyhydroxy Acid Cleanser Evaluation

In order to determine whether the addition of polyhydroxy acid in the composition could enhance aesthetics and tolerability of a formulation containing oxidizing agents, a panel of twelve volunteers compared two test wash-off cleanser formulations. The first formulation contained 7.5% by weight benzoyl peroxide plus 4% gluconolactone, and the second formulation contained 7.5% by weight benzoyl peroxide plus 10% gluconolactone. After using the cleansers for a controlled period of time, the formulation containing benzoyl peroxide plus 10% gluconolactone was preferred over the formulation containing 4% gluconolactone because it more effectively removed dirt and oil, and did not cause as much erythema as the 4% formulation. Nearly 80% of the panel selected the 10% gluconolactone-containing formulation in a forced preference test. The results of this direct comparison demonstrate the utility of including a polyhydroxy acid in a wash-off formulation containing the oxidizing drug benzoyl peroxide.

### EXAMPLE 7

### Benzoyl Peroxide+Polyhydroxy Acid Cleanser—Irritation Assessment

In order to evaluate the irritation potential of 7.5% benzoyl peroxide plus 10% polyhydroxy acid containing cleansers, four formulation prototypes were tested versus a mild, commercially available cleanser that did not contain benzoyl peroxide in a modified cumulative irritation test.

Six subjects entered into the study, Subjects were not currently taking any medications including anti-inflammatory agents, antibiotics, and/or anti-histamines. Subjects were not pregnant or nursing and were between the ages of 18-70. The subjects had no known allergies to alpha or poly hydroxyacids, sunscreens, or other skin care products. Subjects were instructed not to apply any moisturizers, lotions or any other products to the test area. Subjects were instructed not to use body scrubs or exfoliants, washcloths, loofahs, or sponges on the test area. Subjects were instructed not to swim, use a sauna, or receive sun exposure at tanning salons on their back for the duration of the study. Subjects were permitted to shower, but were instructed to avoid exposing the test area to direct streams of water or excessive soaking.

Prior to the first patch application, the test sites were wiped with 70% isopropyl alcohol and allowed to dry. Using a 1 cc syringe, approximately 0.2 ml of each test material was applied to a patch (TruMed Technologies Inc.) and to the back on either side of the spine. Semi-occlusive patches were used for the products containing benzoyl peroxide for the first 4 days of the study so as to avoid any severe irritation that may occur over the weekend. An occlusive patch was used for the non-benzoyl peroxide containing product at the onset of the study and all products were patched occlusively starting on Monday. The cleansers were diluted to 10% prior to application to simulate in-use conditions.

Initial patching took place on a Thursday. Daily applications and readings took place on Friday and the following week, Monday through Friday, with the last Friday as a reading day only. The patches applied on the first Friday remained in place over the weekend and were removed on Monday. There were a total of 6 readings over an 8-day period. The subjects wore the patches for approximately 24 hours at a time and reported to the lab each weekday to have the patches removed and to have fresh patches applied.

The sites were marked to ensure the continuity of repetitive patch applications. The subjects were instructed to take care not to wash off the marks above each patch.

Prior to re-application, the test sites were evaluated using the following scoring scale:

0=No visible reaction

+(0.5)=Barely perceptible or spotty erythema

1=Mild erythema covering most of the test site

2=Moderate erythema, possible presence of mild edema

3=Marked erythema, edema

4=Severe erythema, edema, vesiculation, bullae, and/or ulceration

If at any time during the study a test site exhibited a score of 3 or greater, the application of the test material to the site was discontinued. The site was assigned the 3 or greater score for the duration of the test. If a subject could not tolerate a test material, the patch was removed at the time of complaint and that score was assigned for the duration of the test.

Total cumulative irritation scores for the four benzoyl peroxide+polyhydroxy acid containing cleansers were between 29.5 and 34.5 out of a maximum possible score of 108. Three of four prototype cleansers caused a single 3 score on the last day of grading; the fourth prototype caused a maximum score of 2.5. The non-irritating control cleanser scored 18. The benzoyl peroxide plus polyhydroxy acid cleansers were relatively well tolerated under the exaggerated conditions of occlusion.

### EXAMPLE 8

### Benzoyl Peroxide+Polyhydroxy Acid Cleanser—Irritation Assessment

A 14-day cumulative irritation study was conducted on 27 healthy subjects to evaluate irritation potential of a prototype cleanser containing 5% benzoyl peroxide+10% gluconolactone; a prototype cleanser containing 7.5% benzoyl peroxide+10% gluconolactone; two control benzoyl peroxide containing foaming cleansers without polyhydroxy acids, at a lower strength (4% benzoyl peroxide) and higher strength (8% benzoyl peroxide); and the controls: 0.9% sodium chloride solution (saline) and no treatment. Cleansers were diluted with water to a concentration of 10% to simulate wash off conditions. All test products were applied under occlusion on visits 1 to 4 and were converted to open patch (i.e, no patch) on visits 5 through 10 due to observed moderate skin irritation with some of the tested formulations. Skin irritation scores were collected at 10 observation time points on weekdays only over the 14-day testing period.

Prior to re-application, the test sites were evaluated using the following scoring scale:

0=No visible reaction

+(0.5)=Barely perceptible or spotty erythema

1=Mild erythema covering most of the test site

2=Moderate erythema, possible presence of mild edema

3=Marked erythema, edema

4=Severe erythema, edema, vesiculation, bullae, and/or ulceration

If at any time an evaluation score of 3 was given, product application was discontinued and a score of 3 was assigned for the duration of the study. The prototype 7.5% benzoyl peroxide+10% gluconolactone cleanser was significantly less irritating (score 358) than the high strength (8%) comparator benzoyl peroxide foaming cleanser (score 706). There was a trend toward less irritation (not significant) versus the low strength (4%) benzoyl peroxide foaming cleanser (score 703). The 5% benzoyl peroxide+10% gluconolactone cleanser scored lower (score 474.5) than both the 4% (score 703) and 8% (score 706) benzoyl peroxide cleanser controls, however the results were not significant. The controls: 0.9% sodium chloride solution (saline) and no treatment were significantly less irritating than the benzoyl peroxide containing formulations as expected.

### EXAMPLE 9

### Benzoyl Peroxide+Polyhydroxy Acid Leave-On Treatment—Irritation Assessment

In order to evaluate the irritation potential of two benzoyl peroxide plus 10% polyhydroxy acid containing leave-on treatments. One contained 5% benzoyl peroxide and one contained 7.5% benzoyl peroxide in prototype lotions. The prototypes were tested versus two leading, commercially available treatments that contained 10% benzoyl peroxide in a modified cumulative irritation test.

Six subjects entered into the study. Subjects were not currently taking any medications including anti-inflammatory agents, antibiotics, and/or anti-histamines. Subjects were not pregnant or nursing and were between the ages of 18-70. The subjects had no known allergies to alpha or poly hydroxyacids, sunscreens, or other skin care products. Subjects were instructed not to apply any moisturizers, lotions or any other products to the test area. Subjects were instructed not to use body scrubs or exfoliants, washcloths, loofahs, or sponges on the test area. Subjects were instructed not to swim, use a sauna, or receive sun exposure at tanning salons on their back for the duration of the study. Subjects were permitted to shower, but were instructed to avoid exposing the test area to direct streams of water or excessive soaking.

Prior to the first patch application, the test sites were wiped with 70% isopropyl alcohol and allowed to dry. Using a 1 cc syringe, approximately 0.2 ml of each test material was applied to a patch (TruMed Technologies Inc.) and to the back on either side of the spine. Initial patching took place on a Thursday. Semi-occlusive patches were used for the first 4 days of the study so as to avoid any severe irritation that may occur over the weekend, and all products were patched occlusively starting on Monday.

Initial patching took place on a Thursday. Daily applications and readings took place on Friday and the following week, Monday through Friday, with the last Friday as a reading day only. The patches applied on the first Friday remained in place over the weekend and were removed on Monday. There were a total of 6 readings over an 8-day period, The subjects wore the patches for approximately 24 hours at a time and reported to the lab each weekday to have the patches removed and to have fresh patches applied.

The sites were marked to ensure the continuity of repetitive patch applications. The subjects were instructed to take care not to wash off the marks above each patch.

Prior to re-application, the test sites were evaluated using the following scoring scale:

0=No visible reaction

+(0.5)=Barely perceptible or spotty erythema

1=Mild erythema covering most of the test site

2=Moderate erythema, possible presence of mild edema

3=Marked erythema, edema

4=Severe erythema, edema, vesiculation, bullae, and/or ulceration

If at any time during the study a test site exhibited a score of 3 or greater, the application of the test material to the site was discontinued. The site was assigned the 3 or greater score for the duration of the test. If a subject could not tolerate a test material, the patch was removed at the time of complaint and that score was assigned for the duration of the test.

The total cumulative irritation scores for the two benzoyl peroxide+polyhydroxy acid containing treatments were nearly equivalents, scoring 21.5 and 20 for the 7.5% and 5% benzoyl peroxide plus 10% gluconolactone formulations respectively. The 10% benzoyl peroxide formulations scored 33 and 25.5. The 5% and 7.5% benzoyl peroxide plus polyhydroxy acid treatments were tolerated well under the condition of this study. There is no advantage pertaining to irritation potential in choosing a lower strength (5%) benzoyl peroxide plus polyhydroxy acid treatment. With the addition of polyhydroxy acid, the irritation potential of the test formulations remains lower than commercially available benchmarks.

### EXAMPLE 10

### Benzoyl Peroxide+Polyhydroxy Acid Treatment—Irritation Assessment

A 14-day cumulative irritation study was conducted on 27 healthy subjects to evaluate the irritation potential associated with a commercially available 8% benzoyl peroxide gel, a commercially available 4% benzoyl peroxide gel, a prototype 7.5% benzoyl peroxide+10% gluconolactone lotion and controls: 0.9% sodium chloride solution (saline) or no treatment. All test products were applied under occlusion on visits 1 to 4, on open skin without a patch on visit 5, and under semi-occlusion on visits 6 to 10. Product application was changed to manage the rapid development of irritation with some of the tested formulations. The 8% benzoyl peroxide gel was directionally more irritating than the 4% benzoyl peroxide gel and 7.5% benzoyl peroxide+10% gluconolactone. Application of 0.9% sodium chloride solution (saline) and no treatment were significantly less irritating than the three benzoyl peroxide formulations.

### EXAMPLE 11

### Benzoyl Peroxide+Polyhydroxy Acid Treatment on Pads—Irritation Assessment

An evaluation of a prototype lotion containing 5% benzoyl peroxide+10% gluconolactone delivered using individual pads was carried out on 58 volunteers. Subjects applied the product to cleansed facial skin in two separate applications. Greater than 80% of the subjects rated the prototype to be mild, gentle, non-stinging and non-burning on skin. The benzoyl peroxide treatment with polyhydroxy acid was non-irritating when applied with a pad.

### EXAMPLE 12

### Benzoyl Peroxide+Polyhydroxy Acid Cleanser Evaluation—Vehicle Assessment

In order to evaluate the effect of vehicle modifications on product preference, a panel of five volunteers compared two test wash-off cleanser formulations. The first formulation contained 7.5% benzoyl peroxide plus 10% gluconolactone without dimethicone, and the second formulation contained 7.5% benzoyl peroxide plus 10% plus 3% dimethicone. The overall opinion of the product containing 3% dimethicone was statistically preferred (p<0.05) over the formulation without dimethicone.

While the invention has been described with reference to particularly preferred examples and embodiments, those skilled in the art will appreciate that various modifications may be made to the invention without departing from the spirit and scope thereof.

What is claimed is:

1. A composition comprising a) an oxidative pharmaceutical drug and b) a polyhydroxy acid or polyhydroxy lactone.

2. The composition of claim 1, wherein the polyhydroxy acid or polyhydroxy lactone is selected from the group consisting of gluconic acid, gluconolactone, ribonic acid, ribonolactone, galactonic acid, galactonolactone, glucoheptonic acid, glucoheptonolactone, glucuronic acid, glucuronolactone, galacturonic acid, galacturonolactone, glucaric acid, glucarolactone, galactaric acid and galactarolactone.

3. The composition of claim 1, wherein the polyhydroxy acid or polyhydroxy lactone is an aldonic acid or aldonolactone comprising the following structure:
R(CHOH)ₙCHOH COOH
where R is H or an alkyl group and n is an integer from 1-9.

4. The composition of claim 1, wherein the polyhydroxy acid and polyhydroxy lactone is an aldaric acid or aldarolactone comprising the following structure:
HOOC(CHOH)ₙCHOH COOH
where n is an integer from 1-9.

5. The composition of claim 1, wherein the polyhydroxy acid and polyhydroxy lactone is an alduronic acid or alduronolactone comprising the following structure:
HOOC(CHOH)ₙCHOH CHO
where n is an integer from 1-9.

6. The composition of claim 1, wherein the oxidative pharmaceutical drug is a peroxide, peracid, or superoxide.

7. The composition of claim 1, wherein the oxidative pharmaceutical drug is selected from the group consisting of benzoyl peroxide, carbamide peroxide, coal tar, glutathione, hydrogen peroxide, iodine, juniper tar, lipoic acid, NAD⁺, NADP⁺, nitrogen oxide, oxygen, pine tar, potassium permanganate, povidone-iodine, perbenzoic acid, sulfur, sulfur dioxide, sodium borate, sodium perborate, shale tar, ubiquinone and wood tar.

8. The composition of claim 1, wherein the oxidative pharmaceutical drug is benzoyl peroxide and the polyhydroxy lactone is gluconolactone.

9. The composition of claim 1, wherein the concentration of the oxidative pharmaceutical drug ranges from about 0.1% to about 30% by weight, based on the total weight of the composition.

10. The composition of claim 1, wherein the concentration of the polyhydroxy acid or polyhydroxy lactone ranges from about 0.1% to about 50% by weight, based on the total weight of the composition.

11. The composition of claim 1, further comprising an additional pharmaceutical or topical agent for synergetic or synergistic effect.

12. A method for treating or preventing cosmetic conditions or dermatological disorders, comprising topically applying an effective amount of a composition comprising a) an oxidative pharmaceutical drug and b) a polyhydroxy acid or polyhydroxy lactone.

13. The method of claim 12, wherein said cosmetic conditions or dermatological disorders are selected from the group consisting of disturbed keratinization, defective syntheses of dermal components, and changes associated with aging of skin, nail and hair; and those indications which include dryness or loose of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; eczema; psoriasis; itchy scalp and skin; pruritus; warts; herpes; age spots; lentigines; melasmas; blemished skin; hyperkeratoses; hyperpigmented skin; abnormal or diminished syntheses of collagen, glycosatinoglycans, proteoglycans and elastin as well as diminished levels of such components in the dermis; stretch marks; skin lines; fine lines; wrinkles; thinning of skin, nail plate and hair, skin thickening due to elastosis of photoaging, loss or reduction of skin, nail and hair resiliency, elasticity and recoilability; lack of skin, nail and hair lubricants and luster; dull and older-looking skin, nail and hair; and fragility and splitting of nail and hair.

14. The method of claim 12, wherein the polyhydroxy acid or polyhydroxy lactone is selected from the group consisting of gluconic acid, gluconolactone, ribonic acid, ribonolactone, galactonic acid, galactonolactone, glucoheptonic acid, glucoheptonolactone, glucuronic acid, glucuronolactone, galacturonic acid, galacturonolactone, glucaric acid, glucarolactone, galactaric acid and galactarolactone.

15. The method of claim 12, wherein the polyhydroxy acid or polyhydroxy lactone is an aldonic acid or aldonolactone comprising the following structure:
R(CHOH)ₙCHOH COOH
where R is H or alkyl group and n is an integer from 1-9.

16. The method of claim 12, wherein said polyhydroxy acid or polyhydroxy lactone is an aldaric acid or aldarolactone comprising the following structure:
HOOC(CHOH)ₙCHOH COOH
where n is an integer from 1-9.

17. The method of claim 12, wherein said polyhydroxy acid or polyhydroxy lactone is an alduronic acid or alduronolactone comprising the following structure:
HOOC(CHOH)ₙCHOH CHO
where n is an integer from 1-9.

18. A method of making a composition for topical administration comprising a) an oxidative pharmaceutical drug and b) a polyhydroxy acid or polyhydroxy lactone, wherein the method comprises:
preparing a solution comprising the polyhydroxy acid or polyhydroxy lactone,
preparing a dispersion comprising the oxidative pharmaceutical drug, wherein the particle size of the oxidative pharmaceutical drug is reduced to an appropriate distribution so that the dispersion contains minimal grittiness and minimal discernable feel when applied to the skin, and
mixing the solution comprising the polyhydroxy acid or polyhydroxy lactone with the oxidative pharmaceutical drug dispersion.

19. The method of claim 18, wherein the particle size of the oxidative pharmaceutical drug is reduced by wet milling, roller milling, crushing, or other typical methods of reducing particle sizes.

20. The method of claim 18, wherein preparing a solution comprising the antioxidant polyhydroxy acid or polyhydroxy lactone further comprises heating the solution and then cooling the solution prior to mixing the solution with oxidative pharmaceutical drug dispersion.
